(19) United States
(12) Patent Application Publication
(10) Pub. No.: US 2026/0004879 A1
(43) Pub. Date: Jan. 1, 2026

(54) SYMBOL-LINKER STORAGE ENCODING SCHEME

(71) Applicant: Seagate Technology LLC, Fremont, CA (US)

(72) Inventors: Sriram CHARI, Shakopee, MN (US); Anil J. REDDY, Minneapolis, MN (US); Mengdi BAO, Shakopee, MN (US)

(21) Appl. No.: 18/759,673

(22) Filed: Jun. 28, 2024

(51) Int. Cl. G16B 30/10; G16B 50/30

(52) U.S. Cl. CPC G16B 30/10; G16B 50/30

(57) ABSTRACT

A system may receive, in a data read request, a DNA subsection identifier identifying a subsection of a synthesized DNA strand. The system may identify, in the subsection of the DNA strand: a first linker nucleotide subsequence (L1S) corresponding to a first end section at which the subsection was synthesized to an anterior subsection of the synthesized DNA strand, and a central nucleotide subsequence (CS) corresponding to at least a part of a central section that separates the first end section from a second end section of the subsection at which subsection was synthesized to a posterior subsection of the synthesized DNA strand. The system may determine, based on one or more nucleotides of the CS and one or more nucleotides of the L1S, data responsive to the data read request. The system may provide, responsive to the data read request, the data.

### BACKGROUND

There is always a desire for more data storage and increased speed of writing to, and reading from that storage, as well as a desire for reduced cost for the stored data.

DNA is an emerging technology for data storage. DNA enables a large amount of data to be stored in a small volume. In certain DNA-based storage methods, DNA is synthesized using oligonucleotides (“oligos”). Oligos are prefabricated, synthesized DNA strands that are stored in reservoirs. The nucleotides (e.g., A, C, G, T; where “A” refers to adenine, “C” refers to cytosine, “G” refers to guanine, and “T” refers to thymine) of the synthesized DNA strand represent the encoded data.

### SUMMARY

This disclosure is directed to encoding data in DNA synthesized from pre-prepared oligos.

In some aspects, the techniques described herein relate to a method, including receiving, in a data read request, a DNA subsection identifier identifying a subsection of a synthesized DNA strand; identifying, in the subsection of the DNA strand: a first linker nucleotide subsequence (L1S) corresponding to a first end section at which the subsection was synthesized to an anterior subsection of the DNA strand, and a central nucleotide subsequence (CS) corresponding to at least a part of a central section that separates the first end section from a second end section of the subsection at which subsection was synthesized to a posterior subsection of the DNA strand; determining, based on one or more nucleotides of the CS and one or more nucleotides of the L1S, data responsive to the data read request; and providing, responsive to the data read request, the data.

In some aspects, the techniques described herein relate to a system, including: one or more hardware processors; a request interface, the request interface being executable by the one or more hardware processors and configured to receive, in a data read request, a DNA subsection identifier identifying a subsection of a synthesized DNA strand; an interpreter executable by the one or more hardware processors and configured to identify, in the subsection of the synthesized DNA strand: a L1S corresponding to a first end section at which the subsection was synthesized to an anterior subsection of the synthesized DNA strand, and a CS corresponding to at least a part of a central section that separates the first end section from a second end section of the subsection at which subsection was synthesized to a posterior subsection of the synthesized DNA strand, and determine, based on one or more nucleotides of the CS and one or more nucleotides of the L1S, data responsive to the data read request; and wherein the request interface is further configured to provide, responsive to the data read request, the data.

In some aspects, the techniques described herein relate to one or more tangible processor-readable storage media embodied with instructions for executing on one or more processors and circuits of a computing device a process for accessing data stored in a DNA strand, the process including: receiving, in a data read request, a DNA subsection identifier identifying a subsection of a synthesized DNA strand; identifying, in the subsection of the DNA strand: a L1S corresponding to a first end section at which the subsection was synthesized to an anterior subsection of the DNA strand, and a CS corresponding to at least a part of a central section that separates the first end section from a second end section of the subsection at which subsection was synthesized to a posterior subsection of the DNA strand; and determining, based on one or more nucleotides of the CS and one or more nucleotides of the L1S, data responsive to the data read request; and providing, responsive to the data read request, the data.

Other systems and methods are also described herein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. These and various other features and advantages will be apparent from a reading of the following detailed description.

### BRIEF DESCRIPTION OF THE DRAWING

The described technology is best understood from the following Detailed Description describing various implementations read in connection with the accompanying drawing.

FIG. 1A is a schematic rendering of a first linker section oligo.

FIG. 1B is a schematic rendering of a symbol section oligo.

FIG. 1C is a schematic rendering of a second linker section oligo.

FIG. 1D is a schematic rendering of a first DNAzyme.

FIG. 1E is a schematic rendering of a second DNAzyme.

FIG. 2 is a schematic rendering of two DNAzymes linking a symbol section oligo and two linker section oligos.

FIG. 3 is a schematic rendering of a DNA strand formed from the symbol section oligo and two linker section oligos of FIG. 2.

FIG. 4 is a schematic rendering of an example of steps for forming a DNA strand from a DNA strand of FIG. 3 via enzyme assembly.

FIG. 5A illustrates an intermediate DNA strand encoding data using an encoding scheme using nucleotides of the symbol section only.

FIG. 5B illustrates the intermediate DNA strand of FIG. 5A encoding data using encoding schemes involving nucleotides of one or more linker sections, according to the described technology.

FIG. 6 provides further details describing a first scheme illustrated in FIG. 5B to encode data according to the described technology.

FIG. 7 provides further details describing a second scheme illustrated in FIG. 5B to encode data according to the described technology.

FIG. 8 provides further details describing a third scheme illustrated in FIG. 5B to encode data according to the described technology.

FIG. 9 depicts a process for accessing data stored in a DNA strand that encodes data in a symbol section and in at least one linker section.

FIG. 10 depicts an integrated circuit on which the process described in FIG. 9 may be performed.

FIG. 11 illustrates an example computing device 1100 for use in implementing the described technology.

### DETAILED DESCRIPTION

In certain DNA-based storage schemes, pre-prepared oligos may be synthesized (e.g., using deoxyribozymes (“DNAzymes”)) into intermediate DNA strands. Intermediate DNA strands may be selected from a library of intermediate DNA strands and the selected intermediate DNA strands may be synthesized into larger DNA strands to encode data. Such DNA-based storage schemes select from a first library of oligos that are referred to as “symbol sections” (e.g., motif sections) and a second library of oligos that are referred to as “linker sections.” A symbol section is joined to a linker section, which is joined to another symbol section, which is joined to another linker section, etc. in a symbol-by-symbol approach, also known as a motif-by-motif approach. To decrease the time to synthesize longer chains, larger starting oligos (e.g., a starting oligo that includes a symbol section joined on either end to a respective linker section) can be used in the libraries. Such DNA-based storage schemes form a DNA strand using multiple DNA symbol sections (i.e., at least two, often at least ten, more often at least twenty) from the library, which are combined using linker sections and DNAzymes (or other molecules used to join sections together). Each symbol section typically utilizes two linker sections; one symbol section may be combined with two linker sections to form an intermediate strand, two intermediate strands are combined into a larger strand, etc. To control the connection of the symbol sections, linker sections, and DNAzymes are selected with particular nucleotides at their ends to enable synthesis of a DNA strand with a desired nucleotide sequence in the symbol sections.

However, certain DNA-based storage schemes for DNA strands synthesized from symbol and linker sections only encode data using nucleotide sequences of symbol sections and do not encode data using nucleotide sequences of linker sections. In other words, in some motif-by-motif DNA-based storage schemes, linker sequences are selected merely for their ability to bind with other linker sequences so that data-encoding symbol sections can be combined. Accordingly, some DNA-based storage schemes include a substantial amount of non-coding linker nucleotide sections, which results in unused length/volume of the synthesized DNA storage strands. Further, devices for synthesizing DNA strands using such DNA-based storage schemes require a large number of reservoirs for synthesizing oligos to encode a full spectrum of data (e.g., all possible combinations represented by the symbol section). For example, a motif-by-motif DNA-based storage scheme having symbol sections that encode data using a 4-nucleotide sequence may require a device having two hundred fifty-six (256) reservoirs (e.g., 4×4×4×4=256 for all possible combination of four nucleotides A, T, C, and G), or 128 reservoirs if symbol sequences can be flipped such that the reverse order of nucleotides gives the 4-nucleotide symbol section a different meaning.

The described technology addresses the deficiencies of the certain DNA-based storage schemes that utilize DNA strands synthesized from symbol and linker sections. The encoding schemes of the described technology reduce the length or volume of synthesized DNA required to encode data by encoding data in linker sections in addition to symbol sections of synthesized DNA. Accordingly, the encoding schemes of the described technology, which are based at least in part on nucleotides of linker sections of synthesized DNA in addition to symbol sections, increase the density of stored information on DNA strands compared to DNA strands that are encoded using nucleotides of symbol sections only. In some implementations, the density of stored information using the encoding schemes of the described technology is up to four times greater than previous encoding schemes. The encoding schemes of the described technology also reduce the number of reservoirs (e.g., corresponding to unique types of prefabricated component DNA segments) required in devices for synthesizing DNA strands for storage. In some implementations, the number of required reservoirs required for a device to synthesize DNA strands for encoding a full spectrum of data using the encoding schemes of the described technology is as much as four times less than the number of reservoirs required using the previous encoding schemes.

In the following description, reference is made to the accompanying drawings that form a part hereof and which is shown by way of illustration of at least one specific implementation. The following description provides additional specific implementations. It is to be understood that other implementations are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

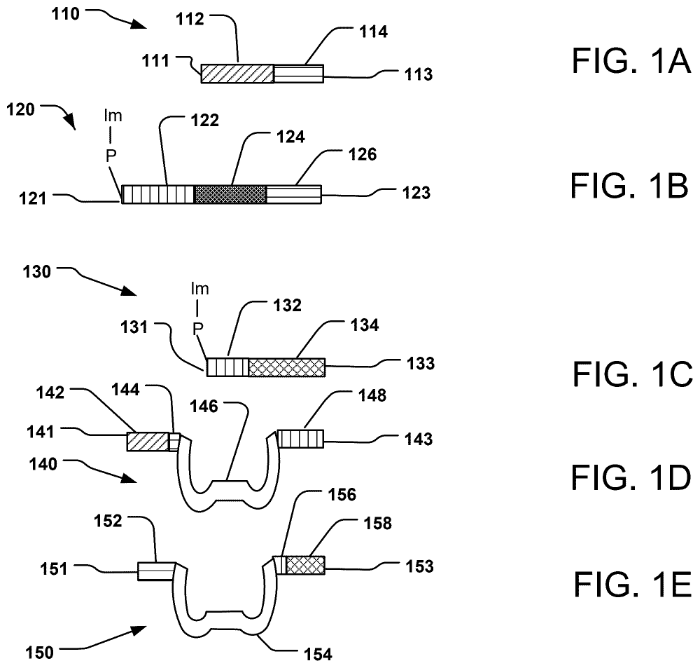

FIGS. 1A through 1E show examples of the components for forming a DNA strand or gene of sufficient length to store usable amounts of data according to this disclosure.

In FIG. 1A a first oligo, referred to herein as a first linker section (e.g., linker section 110), is shown. This linker section 110 is a single-strand, DNA fragment. This first linker section (e.g., linker section 110) is shown with a first sequence subsection 112 at a first end 111 and a second sequence subsection 114 at a second end 113, each of the subsections 112, 114 composed of a plurality of nucleotides.

FIG. 1B shows an oligo referred to herein as a symbol section 120. This symbol section 120 is a single-strand, DNA fragment, typically longer than a linker section (e.g., the linker section 110). This symbol section 120 is shown with a first sequence subsection 122 at a first end 121, a second (center) sequence subsection 124, and a third sequence subsection 126 at a second end 123, each of the subsections 122, 124, 126 composed of a plurality of nucleotides. The first sequence subsection 122 at the first end 121 is an S1 end, and the third sequence subsection 126 at the second end is an S2 end. Additionally, the first sequence subsection 122 is shown with a phosphate-imidazole group, a conventional feature when using certain DNAzyme for synthesis.

The symbol section 120 will usually be composed of a number (e.g., four, five, eight, twelve, or other number) of base nucleotides forming the subsection 124, with the S1 and S2 linking subsections 122, and 126 at each end. These S1 and S2 linking subsections 122, and 126 may have any number of nucleotides, e.g., less than the symbol base subsection 124, about the same, or more. In some embodiments, each of the linking subsections 122, and 126 will have a number (e.g., four, five, six, eight, ten, twelve, or other number) of nucleotides.

FIG. 1C shows another oligo, referred to herein as a second linker section (e.g., linker section 130). Similar to the first linker section (e.g., linker section 110), the second linker section (e.g., linker section 130) is a single-strand, DNA fragment with a first sequence subsection 132 at a first end 131 and a second sequence subsection 134 at a second end 133, each of the subsections 132, 134 composed of a plurality of nucleotides. The first sequence subsection 132 is shown with a phosphate-imidazole group, a conventional feature when using certain DNAzyme for synthesis.

The linker sections (e.g., linker section 110, linker section 130) may be composed of six to 20 nucleotides, with the end nucleotides complementary to either the ends of the symbol section 220 or to ends of a DNAzyme, discussed below.

FIGS. 1D and 1E each show a DNAzyme, specifically, DNAzyme 140 (FIG. 1D) and DNAzyme 150 (FIG. 1E). The DNAzyme 140 has four sequence sections, a first sequence section 142 at a first end 141 of the DNAzyme 140, a second sequence section 144, a third sequence section 146, and a fourth sequence section 148 at the second end 143 of the DNAzyme 140, each of the sections 142, 144, 146, 148 composed of a plurality of nucleotides. The section 146 of the DNAzyme 140 is the E47 sequence whereas the sections 142, 144, and 148 are tailored to the particular application. The sequence section 148 at the second end 143 is complimentary to an S1 end. The DNAzyme described herein may be used for the synthesis of the oligo components (e.g., a symbol section 120 with two adjacent linker sections (e.g., linker section 110 and linker section 130)) and/or synthesis of a DNA strand from the oligo components described herein. However, other methods (e.g., enzyme-based methods other than DNAzymes) may be used instead of or in addition to using DNAzymes.

The example DNAzyme 150 also has four sequence sections, a first sequence section 152 at the first end 151 of the DNAzyme 150, a second sequence section 154, a third sequence section 156, and a fourth sequence section 158 at the second end 153 of the DNAzyme 150, each of the sections 152, 154, 156, 158 composed of a plurality of nucleotides. The section 154 of the DNAzyme 150 is the E47 sequence whereas the sections 152, 156, and 158 are tailored to the particular application. The sequence section 152 at the first end 151 is complimentary to an S2 end.

Together, the linker sections (e.g., linker section 110, linker section 130), the symbol section 120, and DNAzymes (e.g., DNAzyme140, DNAzyme150), or other molecule/enzyme used for DNA synthesis, are part of a system that can be used to form a DNA strand or gene. The linker sections (e.g., linker section 110, linker section 130) are part of a library of linker sections; the symbol section 120 is part of a library of symbol sections; and the DNAzymes (e.g., DNAzyme 140, DNAzyme 150) are part of a library of DNAzymes. Each of the libraries is composed of multiple (e.g., hundreds, thousands) oligos (linker sections, symbol sections) and DNAzymes modified to ligate with the linker sections and the symbol sections.

Although the linker sections (e.g., linker section 110, linker section 130) are shown with two linker subsections 112, 114, and 132, 134, respectively, it is to be understood that additional subsections may be present in one or both linker sections (e.g., linker section 110 and/or linker section 130). Additionally, the symbol section 120 may have more (e.g., four or more) or less (e.g., two) subsections. The example DNAzymes (e.g., DNAzyme 140, DNAzyme 150) have at least three sections, with one of the sections being the catalytic portion, e.g., E47.

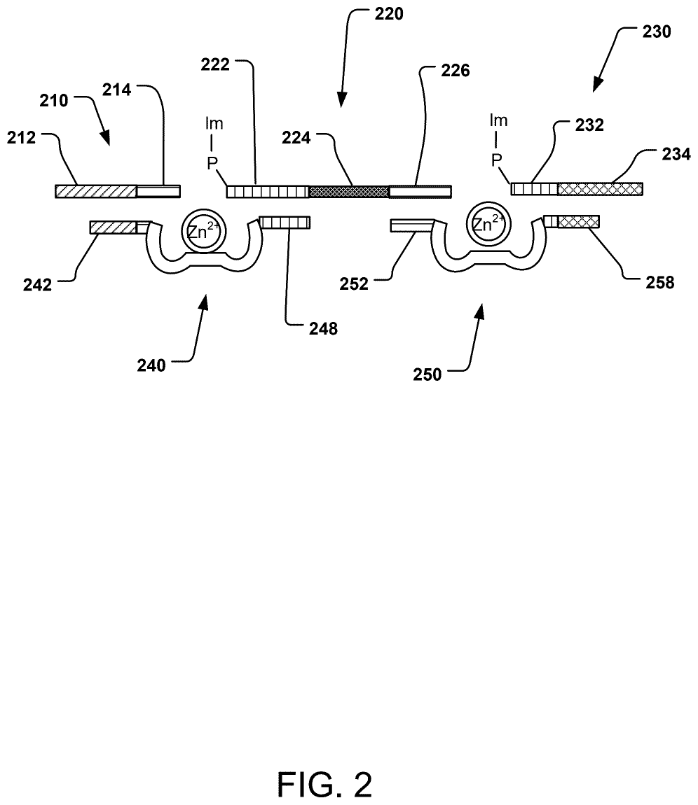

The different patterns in the sequence sections designate different complementary sequences, those that will ligate, or join. FIG. 2 shows the oligos of FIGS. 1A through 1E ligated, in a particular order based on the sequence sections.

In FIG. 2, the first linker section 210 is joined to the S2 first end subsection of the first DNAzyme 240; particularly, the sequence subsection 212 is complementary to and thus ligates with the sequence subsection 242 and the sequence subsection 214 is complementary to and ligates with the subsection 242. At the S1 second end subsection of the DNAzyme 240, the sequence subsection 248 is complementary to and ligates with the subsection 222 of the symbol section 220 (which includes subsection 222, subsection 224, and subsection 226) at the S1 first end subsection 212. The second DNAzyme 250, particularly the sequence subsection 252 at the S2 first end subsection, is complementary to and ligates with the S2 second end subsection 226 of the symbol section 220. At the second end subsection of the DNAzyme 250, the sequence subsection 256 is complementary to and ligates with the subsection 232 of the symbol section 220 and the subsection 258 is complementary to and ligates with the subsection 234 of the symbol section 230.

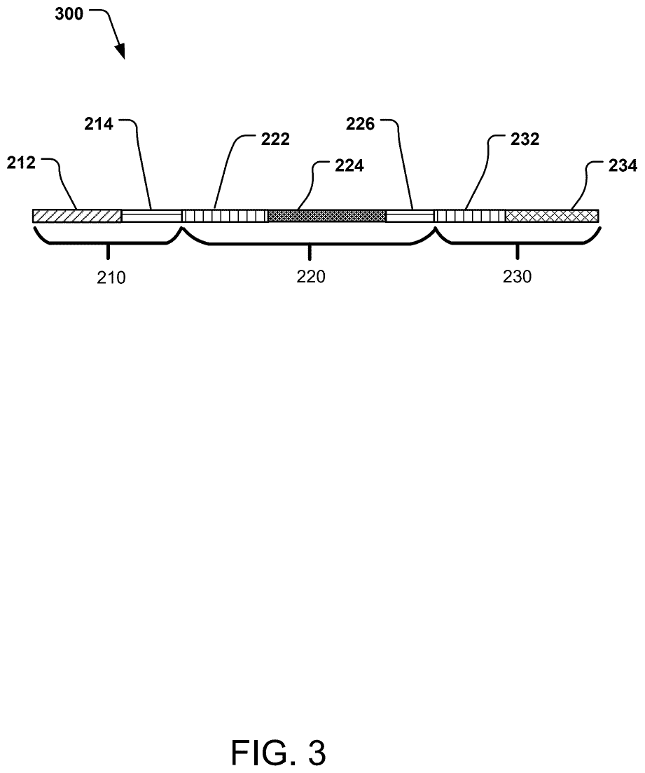

Summarized, the DNAzymes 240, and 250 may be used to attach the linker sections 210, and 230 to the symbol section 220. In such a manner, a single-strand DNA unit (intermediate strand 300), shown in FIG. 3, is formed from the first linker section 210, the symbol section 220, and the second linker section 230. As indicated above, the 3′ end of the symbol section 220 and the second linker section 230, shown as the S1 first end subsection 222 of the symbol section 220 and the first end subsection 232 of the linker section 230, are ‘activated’ ends, activated by phosphate and imidazole before ligation. In some implementations, during ligation, the phosphate and imidazole release and do not appear in the intermediate strand 300. In some implementations, the DNAzymes 240, and 250 are removed by various means, e.g., chemical, or physical methods that can include heat, strand displacement, or conjugation to magnetic beads.

The intermediate strand 300, as formed above, may be faster and less expensive to form than DNA strands ligated using enzymes. By replacing enzymes with DNAzymes, the cost of forming large DNA strands for data storage is greatly reduced. Using DNAzymes also increases the flexibility available during the assembly method. As shown above, DNAzymes can be used to attach linker sections to symbol sections, eliminating the enzymes which can be the most expensive step. Additionally, DNAzymes can be used to assemble multiple intermediate DNA strands, e.g., intermediate strand 300, in downstream steps to form DNA strands or genes having sufficient length to encode usable amounts of data.

After the DNAzymes 240, 250 are used to attach the linker sections 210, 230 to the symbol section 220, an assembly method such as PCR (Polymerized Chain Reaction), Gibson assembly, or another assembly method may be used to assemble the DNA strands (e.g., intermediate strand 300), e.g., via complementary linker sections. DNAzymes may also be used to join the intermediate DNA strands (e.g., intermediate strand 300) together via their linker sections in subsequent steps.

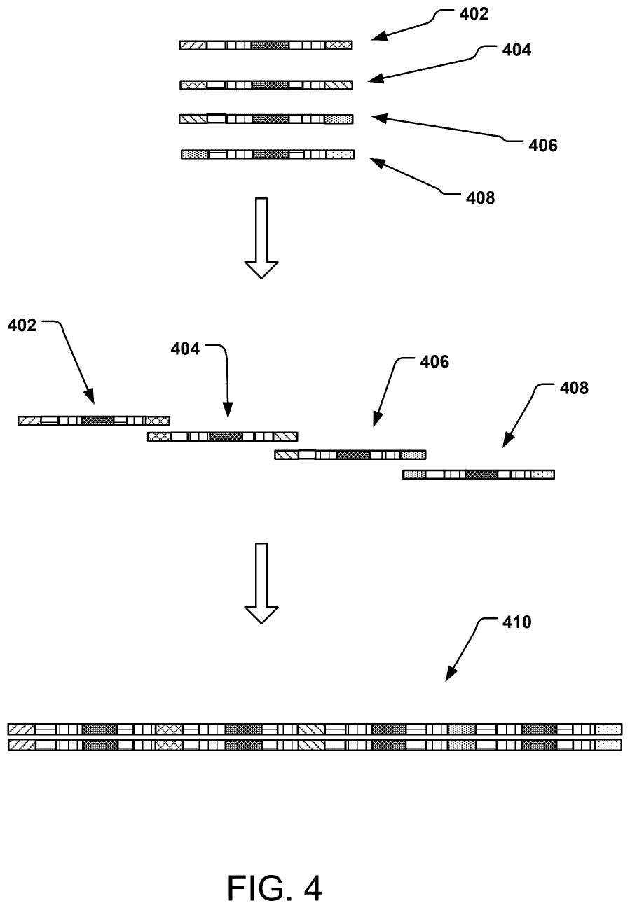

FIG. 4 illustrates, step-wise, an example of the assembly of multiple intermediate DNA strands (e.g., each intermediate strand 300 including a symbol section and two linker sections) into longer DNA strands.

In FIG. 4, four intermediate DNA strands (e.g., intermediate strand 402, intermediate strand 404, intermediate strand 406, intermediate strand 408) are shown. In some implementations, each of the intermediate DNA strands is prepared by the method described above using DNAzymes. The intermediate strands (e.g., intermediate strand 402, intermediate strand 404, intermediate strand 406, intermediate strand 408) can be linked using an assembly PCR, Gibson assembly, or another enzymatic assembly to form a longer strand 410, which is shown as a double strand, due to being formed by an enzyme assembly method.

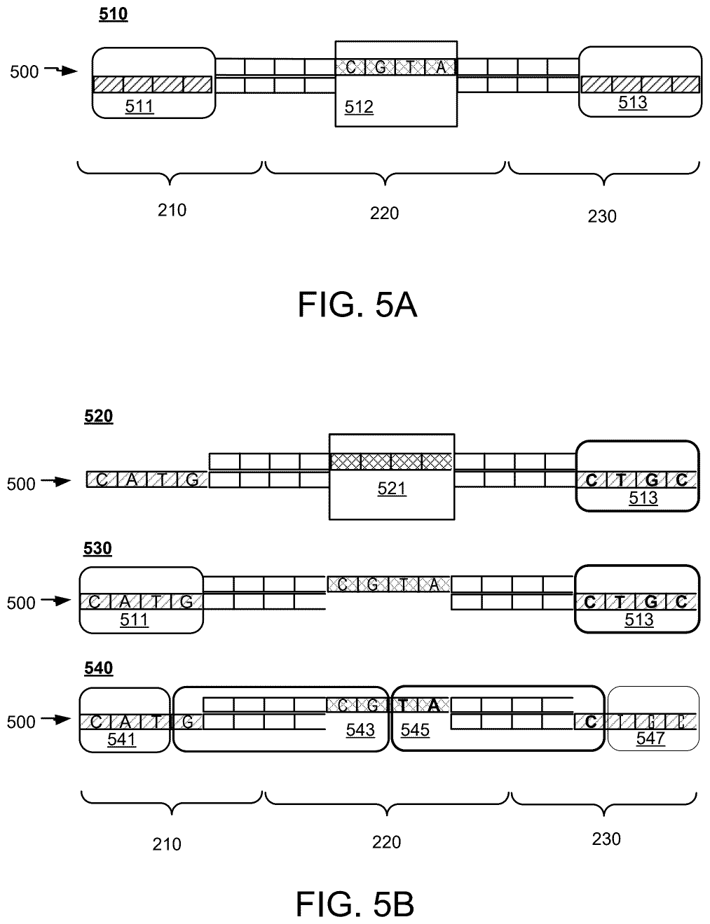

FIG. 5A illustrates an intermediate DNA strand (e.g., intermediate strand 500) encoding data using a particular encoding scheme 510 using nucleotides of the symbol section only. In the particular encoding scheme 510, the nucleotides of symbol section 512 of the symbol section 220 (C, G, T, A) encode data, while nucleotides of linker sections (e.g., linker section 511, linker sections 513) as well as nucleotides of regions overlapping between the linker sections 210, 230 and the symbol section 220 do not encode data. Various encoding methods may be used. For example, each nucleotide may be assigned a bit pattern. In a one-to-one encoding method may represent each nucleotide as a single bit with a value of 0 or 1 (e.g., A, T=1, G, C=0). In a binary encoding method, each of the four possible nucleotides corresponds to a two-bit value, e.g., A=00, C=10, G=01, and T=11. In the binary encoding method, pairs of nucleotides may encode a corresponding binary pattern, as illustrated in Table 1 below:

TABLE 1

| DNA Oligo | Binary |
|---|---|
| AA | 0000 |
| AG | 0001 |
| AC | 0010 |
| AT | 0011 |
| GA | 0100 |
| GG | 0101 |
| GC | 0110 |
| GT | 0111 |
| CA | 1000 |
| CG | 1001 |
| CC | 1010 |
| CT | 1011 |
| TA | 1100 |
| TG | 1101 |
| TC | 1110 |
| TT | 1111 |

Using the example in Table 1 above, AA is 0000; the two base pair oligo stores 4 bits. As the oligo strand lengthens, more bits, bytes, and data can be stored. For example, an oligo that is 8 base pairs long stores 16 bits, or 2 bytes. Using the example in FIG. 5A, an oligo CGTA is 10011100, storing one byte. It is noted that the example in Table 1 is an example of a primitive case and other bit mappings are possible where both the mapping and number of nucleotides per bit are different.

In a ternary encoding method, bits are converted to trits (e.g., ternary digits) and are represented by letters. For example, in the ternary encoding method, A may represent 0, G may represent 1, and T may represent 2. Using the following Table 2, the DNA strand can encode trits based on a value of a previous nucleotide in the sequence and a desired trit value:

TABLE 2

| Previous | 0 | 1 | 2 |
|---|---|---|---|
| T | A | C | G |
| G | T | A | C |
| C | G | T | A |
| A | C | G | T |

For example, when the previous nucleotide in the sequence is C, a following nucleotide G encodes a “0,” a following nucleotide T encodes a “1,” and a following nucleotide A encodes a “2.”

The previously discussed approaches (one-to-one, binary, ternary encoding methods) can either represent data in a bit-by-bit approach or may be combined with lookup tables.

Methods other than the example methods described above may be used to encode data in the encoding scheme 510, which encodes data using one or more nucleotides of the symbol section 220 of the intermediate DNA strand and which does not encode data using the linker sections 210 and 230.

FIG. 5B illustrates the intermediate DNA strand (e.g., intermediate strand 500) of FIG. 5A encoding data using encoding schemes 520, 530, and 540 involving nucleotides of one or more linker sections, according to the described technology. Unlike the encoding scheme 510, the encoding schemes of the described technology (e.g., encoding scheme 520, encoding scheme 530, encoding scheme 540) consider one or more nucleotides of one or more linker sections of the intermediate DNA strand when determining what data is encoded by the intermediate strand 500.

In one implementation, an encoding scheme of the described technology represents data in the linker sections and the symbol section of an intermediate strand 500 using one or more of a one-to-one encoding method, a binary encoding method, or a ternary encoding method. The symbol and linker sections of the intermediate strand 500 are examples and other symbol and linker sections may be used. The nucleotide sequence of the first linker section, the symbol section, and the second linker section may be concatenated (e.g., left to right or right to left) to encode data using a bit-by-bit approach. For example, the first linker section has a nucleotide sequence of CATG, a symbol section has a nucleotide sequence of TACG, and a second linker section has a nucleotide sequence of CTCG. In this example, the concatenated sequence is CATGTACGCTCG which may encode data in one or more of a one-to-one encoding method, a binary encoding method, or a ternary encoding method. Storing data on the linker sections in this manner increases the storage capacity of the intermediate strand 500 compared to the storage capacity of encoding schemes which only encode data using the nucleotide sequence of the symbol section.

In one implementation, encoding scheme 520 encodes data based at least in part on a linker section 513, which identifies a key value of a table, and a symbol section 521 that encodes a subkey value of a sub-table associated with the key value of the table. For example, a second linker section (e.g., linker section 513) represents a unique key value of a lookup table (e.g., a 9×9 lookup table having 81 key values). In some implementations, the symbol section encodes one of a set (e.g., a set of four) of possible sub-key values for the sub-table. The value associated with the sub-key value of the specific sub-table is represented by a specific nucleotide in the symbol section. For example, 81 sets of sub-tables, where each sub-table encodes one of four unique data values, enables a sub-table encoding section 521 (which includes a key value identified by a linker section and a subkey value identified by a portion of the symbol section) to represent one of 256 unique possible values. Other table sizes and sub-table sizes may be used other than the example configuration of a 9×9 (81 item) table including 4-item sub-tables that are described herein. In some implementations, each of the two linker sections represents a respective key value and the symbol section represents a respective subkey value for each of the key values represented by the two linker sections.

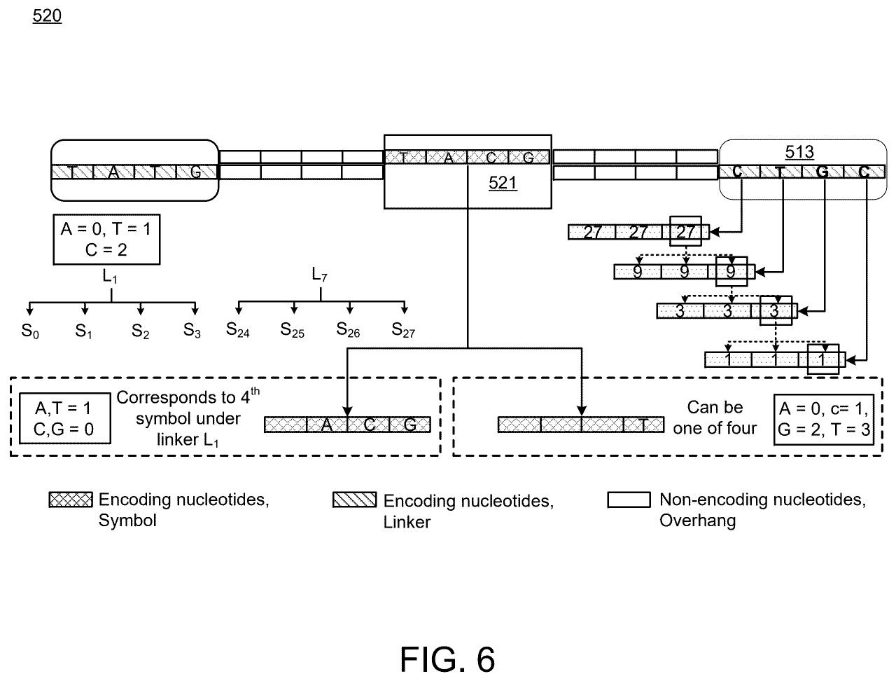

FIG. 6 provides further details describing the method to encode data using encoding scheme 520 of FIG. 5. For example, the linker section 513 represents a unique key value of a 9×9 (81-item) table. For example, the linker section 513 nucleotide sequence is C, A, T, G, where A=0, T=1, and C=2, The first value of the sequence, C, therefore represents “2.” Using the ternary lookup Table 2, the next three values (A, T, G) represent “2,” “2,” and “2,” respectively. As illustrated in FIG. 6, each of these values “2” indicates a third (e.g., where 0 represents a first of three, 1 represents a second of three, and 2 represents a third of three) of three nested hierarchical sets of possible unique key values for the 9×9 table. For example, each of three sets of 27 key values includes a respective three sets of 9 key values. Each of the sets of 9 key values includes a respective three sets of 3 key values. Each of the sets of 3 key values includes a respective three sets of 1 key value each. Accordingly, “2, 2, 2, 2” encoded by the linker section 513 represents the third unique key value within the third set of three key values, within the third set of 9 key values within the third set of 27 key values, as indicated in FIG. 6. The key value identified by linker section 513 is therefore unique key value of a set of 81 possible unique key values. The example of FIG. 6 includes a second linker section, which, in some implementations, identifies a second key value. For example, z starting number comes from the gray box in FIG. 6, which is on the left (A=0, T=1, C=2). For example, in the sequence TAT G, the first nucleotide is a T, which corresponds to “1.” For the subsequent nucleotides, the ternary encoding table determines the value. For example, the second nucleotide in the sequence is A and, according to the ternary table, because the previous nucleotide is a T and the next (or current) necleotide is an A, the value of “A” corresponds to “0.” The same process can be used to determine the remaining values of T and G in the example sequence T A T G. Accordingly, T A T G represents “1, 0, 2, 2,” which represents the third unique key value within the third set of three key values, within the first set of 9 key values within the third set of 27 key values.

After identifying the key value(s) identified by a linker section(s), a respective subkey value for a table associated with the key value(s) for the respective linker section(s) is identified using the symbol section. For example, a value of the last nucleotide of the symbol section may correspond to one of four possible subkey values (e.g., A=0, C=1, G=2, T=3) for a key value identified by linker section 513. Accordingly, in the example of FIG. 6, the last nucleotide symbol of the symbol section (TACG) is a G, which represents a “2.” Therefore, the sub-key value identified by sub-table encoding section 521 is “2.” In some examples, a value of a specific nucleotide (e.g., a second nucleotide) of the symbol section may correspond to one of four possible subkey values (e.g., A=S0, C═S1, G=S2, T=S3) for a first key value L1 identified by a first linker section and another specific nucleotide (e.g., a last nucleotide) of the symbol section may correspond to one of four possible subkey values (e.g., A=S24, C═S25, G=S26, T=S27) for a second key value L7 identified by a second linker section. Accordingly, in some implementations, the single intermediate strand 500 may identify two values (e.g., by extracting each of the two values from its respective sub-table) with separate key/subkey lookup operations. For example, the two identified key-subkey pairs enable retrieval of two data values associated with those two specific key/subkey pairs.

Using the encoding scheme 520, an information density of 16 bits per 20 nucleotides can be achieved. The 16 bits per 20 nucleotide storage capacity of encoding scheme 520 is two times greater than the storage capacity of 8 bits per strand achievable using a conventional bit-per-bit approach in which the nucleotide sequence across the symbol section is read sequentially (e.g., 4 nucleotide sequence each representing 2 bits). Further, using encoding scheme 520, the number of required unique reservoirs is only 97, whereas symbol-only encoding schemes require 256 unique reservoirs.

In some implementations of the example encoding scheme 520, in addition to identifying subkey value(s) for one or two key values identified by the linker section(s), the symbol section represents further data in a bit-by-bit encoding method, to further increase the data storage capacity of the intermediate strand 500. For example, in the symbol section, a first nucleotide represents a first subkey for the key identified by the first linker section, a fourth nucleotide represents a second subkey for the key identified by the second linker section, and second and third nucleotides encode further data using a one-by-one encoding scheme, a binary encoding scheme, or a ternary encoding scheme. Accordingly, the data values encoded by the intermediate strand 500 may include a first value associated with the first subkey identified by the first nucleotide of the symbol section in a table referenced by the first linker section, a second value associated with the second subkey identified by the fourth nucleotide of the symbol section in a table referenced by the second linker section, and subsequent value(s) encoded by the second and third nucleotides of the symbol section.

Returning to FIG. 5, in one implementation, encoding scheme 530 encodes data based at least in part on a linker section 511, a symbol section, and a linker section 513, which each represent a respective key value of a table associated with a respective sub-table, and a supplemental DNA strand that identifies, for each of the sub-tables, a respective sub-key value. For example, linker section 511 may encode key value 15 (of 81 possible key values of a 9×9 lookup table), the symbol section may encode key value 10, and the linker section 511 may also encode key value 15. In this example, the supplemental DNA strand may identify sub-key values 1, 3, and 2 (where four possible sub-keys 0, 1, 2, 3 exist in each of the 81 sub-tables of the 9×9 lookup table), which correspond to the key values 15, 10, and 15. Other table sizes and sub-table sizes may be used other than the example configuration of a 9×9 (81 item) table including 4-item sub-tables that are described herein.

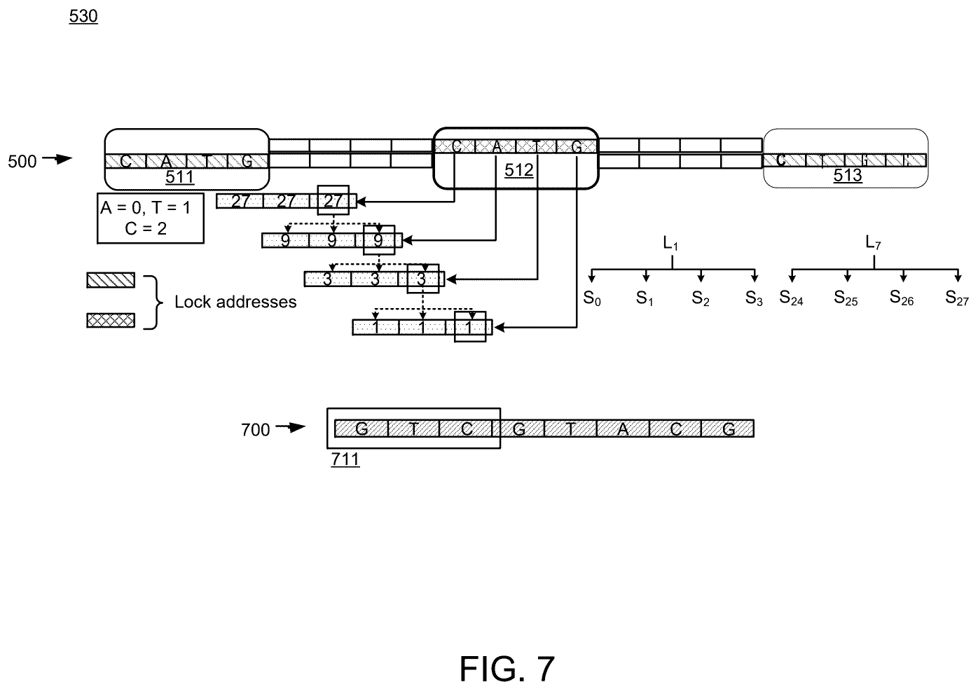

FIG. 7 provides further details describing the method to encode data using encoding scheme 530 of FIG. 5, using an intermediate strand 500 and a supplemental DNA strand 700. For example, the nucleotide sequence of the linker section 511 represents a key value of the 9×9 (81-item) table that is associated with a respective sub-table, the nucleotide sequence of the symbol section 512 represents a key value of the 9×9 (81-item) table that is associated with a respective sub-table, and the nucleotide sequence of the linker section 513 represents a key value of the 9×9 (81-item) table that is associated with a respective sub-table. For example, the symbol section 512 nucleotide sequence is C, A, T, G, where A=0, T=1, and C=2. The first value of the sequence, C, therefore represents “2.” Using the ternary lookup Table 2, the next three values (A, T, G) represent “2,” “2,” and “2,” respectively. As illustrated in FIG. 7, each of these values “2” indicates a third (e.g., where 0 represents a first of three, 1 represents a second of three, and 2 represents a third of three) of three nested hierarchical sets of possible unique key values for the 9×9 lookup table. For example, each of three sets of 27 key values includes a respective three sets of 9 key values. Each of the sets of 9 key values includes a respective three sets of 3 key values. Each of the sets of 3 key values includes a respective three sets of 1 key value each. Accordingly, “2, 2, 2, 2” encoded by the symbol section 512 represents the third unique key value within the third set of three key values, within the third set of 9 key values within the third set of 27 key values, as indicated in FIG. 7. The key value identified by symbol section 512 is, therefore, a key value of a set of 81 possible key values.

After identifying the three respective key values identified by each of the linker section 511, the symbol section 512, and the linker section 513 of the intermediate strand 500, subkey values for a respective sub-table identified by each of the key values are identified based on the nucleotide sequence of the supplemental DNA strand 700. For example, a value of a specific nucleotide of the supplemental DNA strand 700 may correspond to one of four possible subkey values (e.g., A=0, C=1, G=2, T=3) of sub-tables identified by the respective key values referenced by linker section 511, the symbol section 512, and the linker section 513, respectively. In some examples, a value of a specific nucleotide of the supplemental DNA strand 700 may correspond to one of four possible subkey values (e.g., A=S0, C═S1, G=S2, T=S3). Accordingly, in the example of FIG. 7, the first three nucleotide symbols 711 of the supplemental DNA strand 700 encode (e.g., in a bit-by-bit approach) a first sub-key identified by nucleotide G, which represents a “2,” a second sub-key identified by nucleotide T, which represents a “3,” and a third sub-key identified by nucleotide C, which represents a “1.” Accordingly, in the example of FIG. 7, the first linker section (e.g., linker section 511) and the first nucleotide of the supplemental DNA strand 700 together identify a first value, the symbol section 512 and the second nucleotide of the supplemental DNA strand 700 together identify a second value, and the second linker section (e.g., linker section 513) and the third nucleotide of the supplemental DNA strand 700 together identify a third value. Accordingly, in some implementations, the intermediate strand 500 together with the supplemental DNA strand 700 may identify three values with separate key/subkey lookup operations. For example, three identified key-subkey pairs enable retrieval of three data values associated with those three specific key/subkey pairs.

Using the encoding scheme 530, an information density of 32 bits per 23 nucleotides can be achieved The 24 bits per 23 nucleotide storage capacity of encoding scheme 530 is significantly greater than the storage capacity of 8 bits per 20 nucleotides achievable using a conventional symbol linker (motif-by-motif) approach, in which the nucleotide sequence across the symbol section is read sequentially (e.g., 4 nucleotide sequence each representing 2 bits). Further, using encoding scheme 530, the number of required unique reservoirs is only 81, whereas symbol-only encoding schemes require 256 unique reservoirs.

Returning to FIG. 5, in one implementation, encoding scheme 540 encodes data based at least in part on sections 541, 543, 545, and 547 including respective nucleotide sequences distributed across two linker sections and a symbol section. Each of the distributed sections 541, 543, 545, and 547 represents the respective key value of a table associated with a respective sub-table. The nucleotide sequences of the distributed sections 541, 543, 545, and 547, in some implementations, encode their respective key values using a ternary encoding scheme (e.g., see Table 2). Using the ternary encoding scheme ensures that the nucleotides do not repeat, ensuring that later sequencing errors that are caused by repeat nucleotides are avoided.

The encoding scheme 540 also includes a supplemental DNA strand that identifies, for each of the four identified sub-tables, a respective sub-key value. For example, distributed section 541 may encode key value 15 (of 27 possible key values of a 9×3 lookup table), distributed section 543 may encode key value 15, distributed section 545 may encode key value 2, and distributed section 547 may encode key value 4.

In this example, the supplemental DNA strand may identify sub-key values 1, 1, 0 (where three possible sub-keys 0, 1, 2 exist in each of the 27 sub-tables of the 9×3 lookup table), which correspond to the key values 15, 15, 2, and 4. Other table sizes and sub-table sizes may be used other than the example configuration of a 9×3 (27 item) table including 3-item sub-tables that are described herein. For example, 4-item sub-tables may be used, each of which is associated with possible sub-keys 0, 1, 2, and 3. Increasing the number of sub-keys from three to four (or higher) may increase the accuracy of a DNA sequencing process to read the stored data.

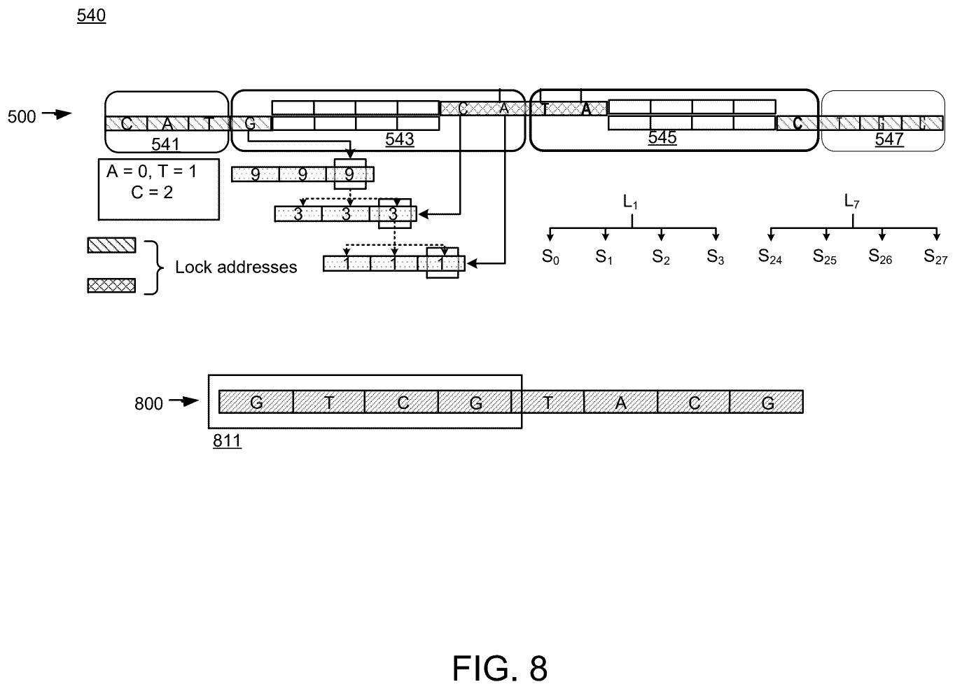

FIG. 8 provides further details describing the method to encode data using encoding scheme 540 of FIG. 5, using an intermediate strand 500 and a supplemental DNA strand 800. The distributed section 541 is a nucleotide sequence including the first three nucleotides of a first linker section, the distributed section 543 is a nucleotide sequence including a fourth nucleotide of the first linker section and the first two nucleotides of the symbol section, the distributed section 545 is a nucleotide sequence including the third and fourth nucleotides of the symbol section and the first nucleotide of the second linker section, and the distributed section 547 is a nucleotide sequence including the final three nucleotides of the second linker section. The distributed section 541 represents a key value of the 9×3 (27-item) table that is associated with a respective sub-table, the distributed section 543 represents a key value of the 9×3 (27-item) table that is associated with a respective sub-table, the distributed section 545 represents a key value of the 9×3 (27-item) table that is associated with a respective sub-table, and the distributed section 547 represents a key value of the 9×3 (27-item) table that is associated with a respective sub-table. For example, distributed section 543 has a nucleotide sequence G, C, and A. The first value of the sequence, G, therefore represents “2.” Using the ternary lookup Table 2, the next two values (C, A) represent “2,” and “2,” respectively. As illustrated in FIG. 8, each of these values “2” indicates a third (e.g., where 0 represents a first of three, 1 represents a second of three, and 2 represents a third of three) of three nested hierarchical sets of possible unique key values for the 9×3 lookup table. For example, each of three sets of 9 key values includes a respective three sets of 3 key values. Each of the sets of 3 key values includes a respective sub-key value. Accordingly, “2, 2, 2” encoded by the distributed section 543 represents the third unique key value within the third set of three key values, within the third set of 9 key values, as indicated in FIG. 7. The key value identified by distributed section 543 is, therefore, a key value of a set of 27 possible key values.

After identifying the four respective key values identified by each of the distributed sections 541, 543, 545, and 547 of the intermediate strand 500, subkey values for a respective sub-table identified by each of the key values are identified based on the nucleotide sequence of the supplemental DNA strand 800. For example, a value of a specific nucleotide of the supplemental DNA strand 800 may correspond to one of three or one of four possible subkey values (e.g., A=0, G/C=1, T=2 or A=0, C=1, G=2, T=3, or other appropriate subkey representation scheme) of sub-tables identified by the respective key values referenced by distributed sections 541, 543, 545, and 547, respectively. Accordingly, in the example of FIG. 8, the first four nucleotide symbols 811 of the supplemental DNA strand 800 encode (e.g., in a bit-by-bit approach) a first sub-key identified by nucleotide G, which represents a “2,” a second sub-key identified by nucleotide T, which represents a “3,” and a third sub-key identified by nucleotide C, which represents a “1,” and a fourth sub-key identified by G, which represents “2.” As previously mentioned, some implementations of the encoding scheme 540 may include only values of 0, 1, and 2 identifying one of three sub-keys. Accordingly, in the example of FIG. 7, the distributed section 541 and the first nucleotide of the supplemental DNA strand 800 together identify a first value, the distributed section 543 and the second nucleotide of the supplemental DNA strand 800 together identify a second value, the distributed section 545 and the third nucleotide of the supplemental DNA strand 800 together identify a third value, and the distributed section 547 and the fourth nucleotide of the supplemental DNA strand 800 together identify a fourth value. For example, four identified key-subkey pairs enable retrieval of four data values associated with those four specific key/subkey pairs.

Using the encoding scheme 540, an information density of 32 bits per 24 nucleotides can be achieved. The storage capacity achieved using encoding scheme 540 is significantly greater than the storage capacity of 8 bits per 20 nucleotides achievable using a conventional symbol linker (motif-by-motif) approach in which the nucleotide sequence across the symbol section is read sequentially (e.g., 4 nucleotide sequences each representing 2 bits). Further, using encoding scheme 540, the number of required unique reservoirs is only 27, whereas symbol-only encoding schemes require 256 unique reservoirs.

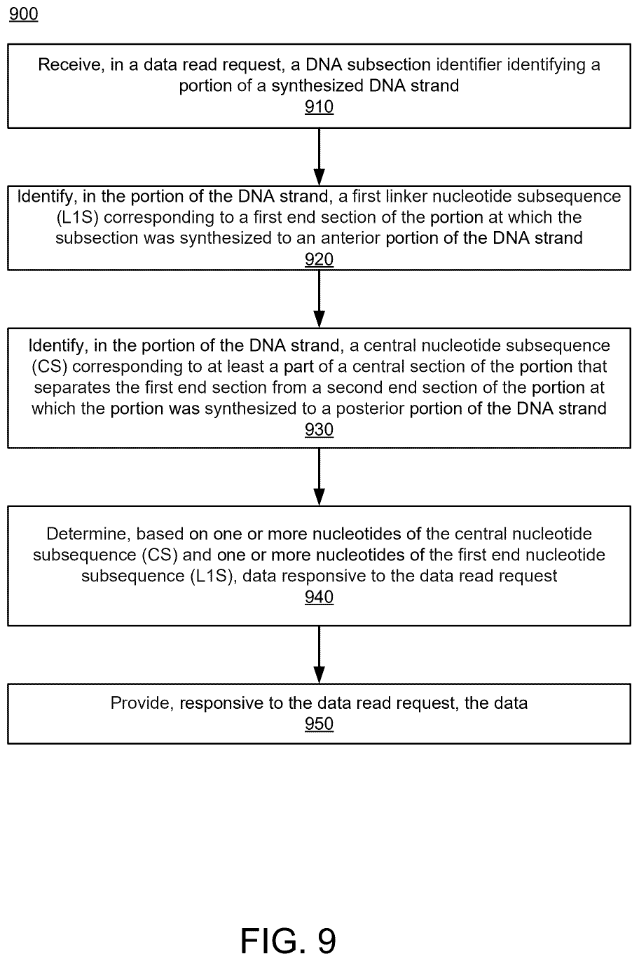

FIG. 9 depicts a process 900 for accessing data stored in a DNA strand that encodes data in a symbol section and in at least one linker section. The example process 900 includes operations 910, 920, 930, 940, and 950.

Operation 910 receives, in a data read request, a DNA subsection identifier identifying a portion of a synthesized DNA strand. DNA strand synthesis methods, as well as reading data encoded on synthesized DNA according to the DNA encoding schemes described herein, can be implemented in any manner, e.g., utilizing various reactors, flasks, beakers, etc. The methods are also particularly suited to be done as a microfluidic lab-on-a-chip process. Lab-on-a-chip is a common term for an integrated circuit (“chip”) on which one or several laboratory functions or chemical reactions are done. One or more operations described herein may be performed using a lab-on-a-chip. In some implementations, the lab-on-a-chip is communicatively coupled to a requesting computing device and the lab-on-a-chip receives the data read request from the requesting computing device. In some implementations the chip receives a nucleotide sequence of the synthesized DNA that is manually sequenced on a different platform. In some implementations, the chip performs DNA sequencing operations on the synthesized DNA and determines the nucleotide sequence itself.

The chip, in some implementations, is no more than a few square centimeters. Labs-on-a-chip handle extremely small fluid volumes (e.g., measured as pico-liters, femto-liters, or other suitable unit) and are often called microfluidic systems. In digital microfluidics, the lab-on-a-chip has a hydrophobic “chip platform” on which fluid droplets (e.g., liquid droplets) can be manipulated by precisely controlled voltage application. The platform may have a cover plate covering the fluidic area. By utilizing the feature of surface tension of the fluid on the platform, the fluid can be precisely moved across the platform by voltage applied to the platform, e.g., in a grid.

Operation 920 identifies, in the portion of the DNA strand, a L1S corresponding to a first end section of the portion at which the subsection was synthesized to an anterior portion of the DNA strand. For example, the L1S is the first linker section of an intermediate strand.

Operation 930 identifies, in the portion of the DNA strand, a CS corresponding to at least a part of a central section of the portion that separates the first end section from a second end section of the portion at which the portion was synthesized to a posterior portion of the DNA strand. For example, the CS is the symbol section of the intermediate strand.

Operation 940 determines, based on one or more nucleotides of the CS and one or more nucleotides of the L1S, data responsive to the data read request. In some implementations, the operation 940 concatenates the symbols of the CS and the L1S into a concatenated sequence and then determines the data represented by the concatenated sequence. The concatenated sequence may represent data according to one or more or according to a combination of one or more of a one-on-one encoding method, a binary encoding method, a ternary encoding method, or any other encoding scheme. In some implementations, the first sequence of nucleotides of the CS represents a key value identifying, in a table, a sub-table and a second sequence of nucleotides of the L1S represents a sub-key value identifying, within the sub-table, a data value. In some implementations, a first sequence of nucleotides of one or more of the L1S and the CS represents a key value identifying, in a table, a sub-table, and one or more nucleotides of a supplemental DNA strand separate from the DNA strand identifies a sub-key value identifying, within the sub-table, a data value.

Operation 950 provides, responsive to the data read request, the data. For example, the operation 950 provides the data to the requesting computing device responsive to the data read request.

For the synthesis of intermediate strands and the synthesis of larger DNA strands from the intermediate DNA strands, the lab-on-a-chip is operably and fluidically connected to the symbol library, with each symbol retained in a well or other liquid storage compartment (e.g., a reservoir). Similarly, the lab-on-a-chip is operably and fluidically connected to the intermediate strand library, with each unique type of intermediate strand (e.g., including a symbol section and two linker sections) retained in a well or other storage compartment.

Using known techniques (e.g., voltage differential on the platform), the intermediate strands are moved on (across) the platform and mixed in the desired steps. All mixing of the oligos (e.g., symbols and linkers) can be done on the platform or a dedicated mixing station may be used for one or more of the joining steps, e.g., utilizing heat and/or agitation. In some implementations, the platform may include a controllable reaction facilitator, such as a UV light source, and/or the final mixing station may include a voltage source, e.g., to align the completed gene to aid in collection.

One suitable (physical) size for a lab-on-a-chip is about 20 mm by 20 mm, which is compatible with an 8-inch wafer and could have 785,000 array elements, each array element having controllable voltage independently applied thereto. In some implementations, each well or other storage compartment for the oligos (symbols or linkers) or DNAzymes is 10× the size of an array element.

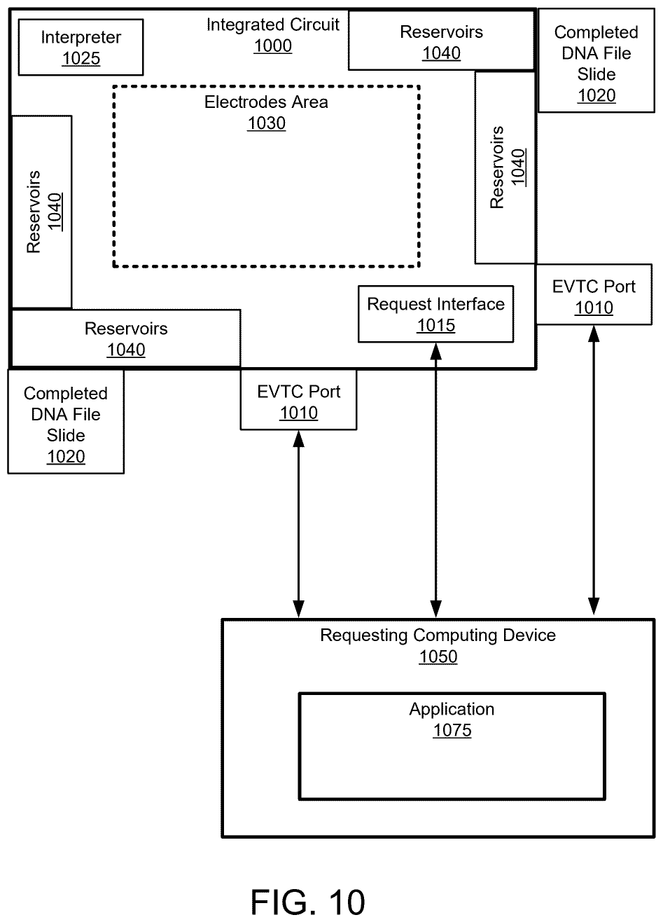

FIG. 10 depicts an integrated circuit 1000 on which one or more processes described herein (e.g., the process described in FIG. 9 to read data encoded on synthesized DNA, processes for synthesizing DNA strands that encode data according to the encoding schemes of the described technology, etc.) may be performed. The integrated circuit 1000 may include one or more electrode voltage and temperature control (EVTC) ports 1010 for controlling conditions for DNA synthesis (e.g., in association with data write operations), and DNA sequencing (e.g., in association with data read operations). The integrated circuit 1000 may include one or more completed DNA file slides 1020. The integrated circuit 1000 creates multiple copies of the DNA strand to be sequenced and compartmentalizes them, in the DNA file slides 1020, for case of access at the time of sequencing. The integrated circuit 1000 may include an electrodes area 1030 (e.g., for assembly of DNA strands and chemical reactions) and reservoirs 1040 for storage of unique intermediate DNA strand components (e.g. one for each type of intermediate strand having a unique combination of sequences in its linker sections and symbol section) and other components (e.g., DNAzymes, enzymes) required for synthesis of DNA strands to encode data according to the encoding schemes of the described technology. The integrated circuit 1000 may be communicatively coupled to a requesting computing device 1050, for example, a user computing device. The integrated circuit 1000 may receive and respond to data read requests and data write requests originating from the computing device 1050 using the request interface 1015. The integrated circuit 1000 may perform, using the interpreter 1025, data read operations (e.g., as described in FIG. 9). In some implementations, the interpreter 1025 of the integrated circuit performs synthesis operations. Synthesis operations may be performed responsive to receiving via the request interface 1015, a data write request including data to be stored, a strand of DNA to encode the data received in a data write request using one or more of the data encoding schemes of the described technology. Synthesis operations may include determining a sequence of intermediate strands needed to synthesize a DNA strand that encodes data received in the data write request using one or more of the encoding schemes of the described technology. Synthesis operations may include communicating with reservoirs 1040 to instruct reservoirs 1040 to provide the identified intermediate strands. Synthesis operations may include changing one or more parameters of the electrodes area 1030 (e.g. a voltage, etc.) to facilitate the synthesis of the DNA strand from the identified intermediate strands. Synthesis operations may include communicating a confirmation response that is responsive to the data write request via the request interface 1015 to the requesting computing device 1050.

The requesting computing device 1050 may include an application 1075 that enables a user to request to initiate read operations to data stored using the integrated circuit and to receive a response including the data read from the synthesized DNA. For example, the data read request may identify a specific synthesized segment of DNA and/or one or more symbol portions or linker portions thereof. The application 1075, in some scenarios, may cause data received responsive to the data read request to be displayed via the user computing device 1050 or via a display device communicatively coupled to the user computing device. User computing device 1050 may receive, from the integrated circuit 1000 responsive to a data write request initiated via the application 1075, a confirmation that a synthetic DNA strand was synthesized to store data specified in the data write request using one or more encoding schemes of the described technology.

In some implementations, one or more operations described herein as being performed by the interpreter 1025 of the integrated circuit 1000 may be performed by a processor of the requesting computing device 1050 and/or one or more operations described herein as being performed by the requesting computing device 1050 may be performed by the interpreter 1025 of the integrated circuit 1000.

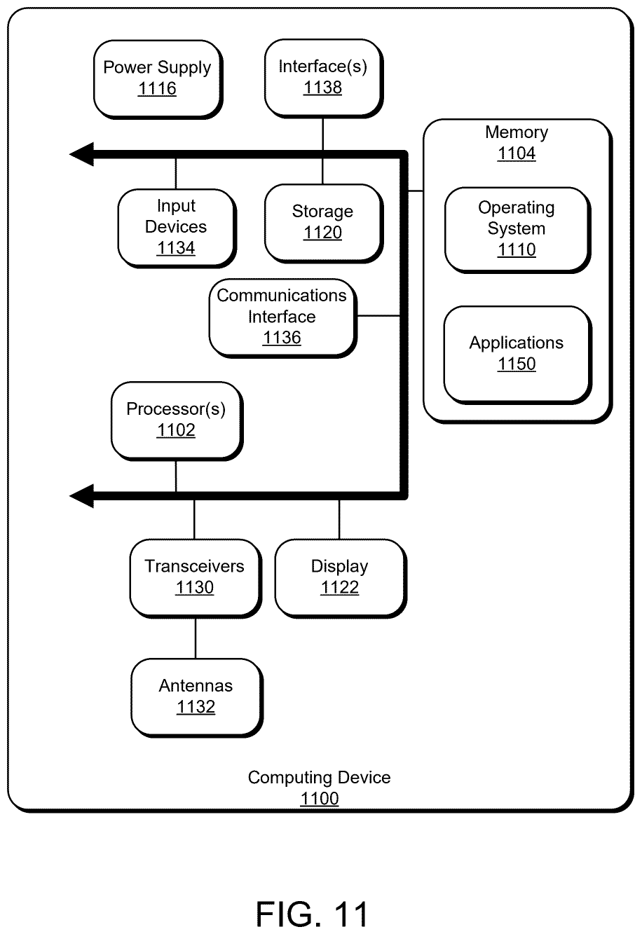

FIG. 11 illustrates an example computing device 1100 for use in implementing the described technology. The computing device 1100 may be a client computing device (such as a laptop computer, a desktop computer, or a tablet computer), a server/cloud computing device, an Internet-of-Things (IoT), any other type of computing device, or a combination of these options. The computing device 1100 includes one or more hardware processor(s) 1102 and a memory 1104. The memory 1104 generally includes both volatile memory (e.g., RAM) and nonvolatile memory (e.g., flash memory), although one or the other type of memory may be omitted. An operating system 1110 resides in the memory 1104 and is executed by the processor(s) 1102. In some implementations, the computing device 1100 includes and/or is communicatively coupled to storage 1120.

In the example computing device 1100, as shown in FIG. 11, one or more software modules, applications 1150, segments, and/or processors, such as an integrated circuit 1000 and one or more components thereof and a requesting computing device 1050 and one or more components thereof. The storage 1120 may store DNA strand identifiers and/or one more identifiers of symbol sections and/or of linker sections of DNA strands, and other data and be local to the computing device 1100 or may be remote and communicatively connected to the computing device 1100.

The computing device 1100 includes a power supply 1116, which may include or be connected to one or more batteries or other power sources, and which provides power to other components of the computing device 1100. The power supply 1116 may also be connected to an external power source that overrides or recharges the built-in batteries or other power sources.

The computing device 1100 may include one or more communication transceivers 1130, which may be connected to one or more antenna(s) 1132 to provide network connectivity (e.g., mobile phone network, Wi-Fi®, Bluetooth®) to one or more other servers, client devices, IoT devices, and other computing and communications devices. The computing device 1100 may further include a communications interface 1136 (such as a network adapter or an I/O port, which are types of communication devices). The computing device 1100 may use the adapter and any other types of communication devices for establishing connections over a wide-area network (WAN) or local-area network (LAN). It should be appreciated that the network connections shown are exemplary and that other communications devices and means for establishing a communications link between the computing device 1100 and other devices may be used.

The computing device 1100 may include one or more input devices 1134 such that a user may enter commands and information (e.g., a keyboard, trackpad, or mouse). These and other input devices may be coupled to the server by one or more interfaces 1138, such as a serial port interface, parallel port, or universal serial bus (USB). The computing device 1100 may further include a display 1122, such as a touchscreen display.

The computing device 1100 may include a variety of tangible processor-readable storage media and intangible processor-readable communication signals. Tangible processor-readable storage can be embodied by any available media that can be accessed by the computing device 1100 and can include both volatile and nonvolatile storage media and removable and non-removable storage media. Tangible processor-readable storage media excludes intangible, transitory communications signals (such as signals per se) and includes volatile and nonvolatile, removable, and non-removable storage media implemented in any method, process, or technology for storage of information such as processor-readable instructions, data structures, program modules, or other data. Tangible processor-readable storage media includes, but is not limited to, RAM, ROM, EEPROM, flash memory or other memory technology, CDROM, digital versatile disks (DVD) or other optical disk storage, magnetic cassettes, magnetic tape, magnetic disk storage, or other magnetic storage devices, or any other tangible medium which can be used to store the desired information and which can be accessed by the computing device 1100. In contrast to tangible processor-readable storage media, intangible processor-readable communication signals may embody processor-readable instructions, data structures, program modules, or other data resident in a modulated data signal, such as a carrier wave or other signal transport mechanism. The term “modulated data signal” means a signal that has one or more of its characteristics set or changed in such a manner as to encode information in the signal. By way of example, and not limitation, intangible communication signals include signals traveling through wired media such as a wired network or direct-wired connection, and wireless media such as acoustic, RF, infrared, and other wireless media.

The above specification and examples provide a complete description of the structure and use of exemplary implementations of the invention. The above description provides specific implementations. It is noted that although not specifically stated, between any of the assembly steps described throughout this description, any additional steps may be added as needed or desired, for example, a PCR amplification step, a purification step, or both. Either of these steps could be performed after a synthesis step (e.g., Gibson assembly step or other synthesis method or protocol). It is to be understood that other implementations are contemplated and may be made without departing from the scope or spirit of the present disclosure. The above-detailed description, therefore, is not to be taken in a limiting sense. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a,” “an,” and “the” encompass implementations having plural referents, unless the content clearly dictates otherwise. As used in this specification and the appended claims, the term “or” is generally employed in its sense including “and/or” unless the content clearly dictates otherwise.

Spatially related terms, including but not limited to, “bottom,” “lower”, “top”, “upper”, “beneath”, “below”, “above”, “on top”, “on,” etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

Since many implementations of the invention can be made without departing from the spirit and scope of the invention, the invention resides in the claims hereinafter appended. Furthermore, structural features of the different implementations may be combined in yet another implementation without departing from the recited claims,

What is claimed is:

1. A method, comprising:
receiving, in a data read request, a DNA subsection identifier identifying a subsection of a synthesized DNA strand;
identifying, in the subsection of the synthesized DNA strand:
a first linker nucleotide subsequence (L1S) corresponding to a first end section at which the subsection was synthesized to an anterior subsection of the synthesized DNA strand, and
a central nucleotide subsequence (CS) corresponding to at least a part of a central section that separates the first end section from a second end section of the subsection at which subsection was synthesized to a posterior subsection of the synthesized DNA strand;
determining, based on one or more nucleotides of the CS and one or more nucleotides of the L1S, data responsive to the data read request; and
providing, responsive to the data read request, the data.

2. The method of claim 1, further comprising:
identifying a second linker nucleotide subsequence (L2S) corresponding to the second end section of the subsection of the synthesized DNA strand,
wherein the data responsive to the data read request is determined based further on at least a portion of the L2S.

3. The method of claim 2, wherein the L1S and the L2S each have four nucleotides, independently, and wherein the CS has twelve nucleotides.

4. The method of claim 2, wherein the portion of the central nucleotide subsequence has four nucleotides.

5. The method of claim 1, wherein determining the data responsive to the data read request comprises determining data corresponding at least to the one or more nucleotides of the CS and the one or more nucleotides of the L1S.

6. The method of claim 2, wherein determining the data responsive to the data read request comprises determining data corresponding at least to the one or more nucleotides of the CS, the one or more nucleotides of the first linker nucleotide subsequence, and the one or more nucleotides of the second linker nucleotide subsequence.

7. The method of claim 5, wherein determining the data associated with at least one symbol comprises extracting the data from a lookup table, wherein the data is a key value associated with a key corresponding to the at least one or more nucleotides of the CS and the one or more nucleotides of the L1S.

8. A system, comprising:
one or more hardware processors;
a request interface, the request interface being executable by the one or more hardware processors and configured to receive, in a data read request, a DNA subsection identifier identifying a subsection of a synthesized DNA strand;
an interpreter executable by the one or more hardware processors and configured to identify, in the subsection of the synthesized DNA strand:
a first linker nucleotide subsequence (L1S) corresponding to a first end section at which the subsection was synthesized to an anterior subsection of the synthesized DNA strand, and
a central nucleotide subsequence (CS) corresponding to at least a part of a central section that separates the first end section from a second end section of the subsection at which subsection was synthesized to a posterior subsection of the synthesized DNA strand, and
determine, based on one or more nucleotides of the CS and one or more nucleotides of the L1S, data responsive to the data read request; and
wherein the request interface is further configured to provide, responsive to the data read request, the data.

9. The system of claim 8, the interpreter being further configured to:
identify a second linker nucleotide subsequence (L2S) corresponding to the second end section of the subsection of the synthesized DNA strand,
wherein the data responsive to the data read request is determined based further on at least a portion of the L2S.

10. The system of claim 9, wherein the L1S and the L2S each have four nucleotides, independently, and wherein the CS has twelve nucleotides.

11. The system of claim 8, wherein central nucleotide subsequence has four nucleotides.

12. The system of claim 8, wherein determining the data responsive to the data read request comprises determining data corresponding at least to the one or more nucleotides of the CS and the one or more nucleotides of the L1S.

13. The system of claim 9, wherein determining the data responsive to the data read request comprises determining data corresponding at least to the one or more nucleotides of the CS, the one or more nucleotides of the first linker nucleotide subsequence, and the one or more nucleotides of the second linker nucleotide subsequence.

14. The system of claim 13, wherein determining the data comprises extracting the data from a lookup table, wherein the data is a key value associated with a key corresponding to the one or more nucleotides of the first linker nucleotide subsequence and one or more nucleotides of the second linker nucleotide subsequence.

15. One or more tangible processor-readable storage media embodied with instructions for executing on one or more processors and circuits of a computing device a process comprising:
receiving, in a data read request, a DNA subsection identifier identifying a subsection of a synthesized DNA strand;
identifying, in the subsection of the synthesized DNA strand:
a first linker nucleotide subsequence (L1S) corresponding to a first end section at which the subsection was synthesized to an anterior subsection of the synthesized DNA strand, and
a central nucleotide subsequence (CS) corresponding to at least a part of a central section that separates the first end section from a second end section of the subsection at which subsection was synthesized to a posterior subsection of the synthesized DNA strand; and
determining, based on one or more nucleotides of the CS and one or more nucleotides of the L1S, data responsive to the data read request; and
providing, responsive to the data read request, the data.

16. The one or more tangible processor-readable storage media of claim 15, the process further comprising:
identifying a second linker nucleotide subsequence (L2S) corresponding to the second end section of the subsection of the synthesized DNA strand,
wherein the data responsive to the data read request is determined based further on at least a portion of the L2S.

17. The one or more tangible processor-readable storage media of claim 16, the process wherein the L1S and the L2S each have four nucleotides, independently, and wherein the CS has four (4) nucleotides.

18. The one or more tangible processor-readable storage media of claim 15, wherein the central nucleotide subsequence has four nucleotides.

19. The one or more tangible processor-readable storage media of claim 15, wherein determining the data responsive to the data read request comprises determining data corresponding at least to the one or more nucleotides of the CS and the one or more nucleotides of the L1S.

20. The one or more tangible processor-readable storage media of claim 16, wherein determining the data responsive to the data read request comprises determining data corresponding at least to the one or more nucleotides of the CS, the one or more nucleotides of the L1S, and the one or more nucleotides of the L2S.
